(19) United States
(12) Patent Application Publication
(10) Pub. No.: US 2005/0019778 A1
(43) Pub. Date: Jan. 27, 2005

(54) Sequential generation of multiple chemiluminescent signals on solid supports

(76) Inventors: John C. Voyta, Sudbury, MA (US); Robert M. Smith, Stow, MA (US); Gary P. Schroth, San Ramon, CA (US); Alison L. Sparks, North Andover, MA (US); Brooks N. Edwards, Cambridge, MA (US); Carolyn Gonzalez, San Mateo, CA (US)

Correspondence Address: Supervisor, Patent Prosecution Services;PIPER RUDNICK LLP, 1200 Nineteenth Street N.W., Washington, DC, 20036-2412, US

(21) Appl. No.: 10/620,332

(22) Filed: Jul. 17, 2003

(51) Int. Cl. C12Q 1/68; G01N 33/53; G01N 33/537; G01N 33/543

(52) U.S. Cl. 435/6; 435/7.92

(57) ABSTRACT

Chemiluminescent assays to determine the presence and/or amount of one or more labeled analytes in a sample are described wherein a solid support is contacted sequentially with first and second chemiluminescent substrates each of which are capable of being activated by an enzyme and the resulting chemiluminescent signals are detected. A plurality of probes are disposed on a surface layer of the solid support in a plurality of discrete areas. Some of the probes are bound to a conjugate of the first enzyme and some of the probes are bound to a conjugate of the second enzyme. The assay can be used to compare biological samples (e.g., mRNA populations from different cells) on the same support surface. Alternatively, one of the chemiluminescent signals generated can be used as a control signal for normalizing the chemiluminescent assay data.

This application is related to U.S. Patent Application Serial No. 10/046,730, filed Jan. 17, 2002, pending, and U.S. patent application Ser. No. 10/050,188, filed Jan. 14, 2002, pending (published as U.S. Patent Application Publication No. US 2002/0110828 A1 on Aug. 15, 2002). This application is also related to U.S. patent application Ser. No. 10/462,742, filed on Jun. 17, 2003, pending. Each of these applications is incorporated herein by reference in its entirety.

### BACKGROUND

1. Technical Field

The subject matter of the present application relates generally to methods of conducting biological assays. More specifically, subject matter of the present application pertains to methods of performing chemiluminescent assays on solid supports wherein two chemiluminescent signals are sequentially generated and detected.

2. Background of the Technology

Microarray technology provides a useful tool for conducting biological assays. A microarray comprises a large number of different probes each of which are immobilized in different discrete areas on a substrate. For nucleic acid assays, the probes can be nucleic acid or oligonucleotide probes. When a sample is contacted with the microarray, molecules in the sample (i.e., target molecules) can hybridize to probes having complementary or substantially complementary sequences. Detection of the position of the hybridized target molecule on the array (e.g., by detecting a label on the target molecule) indicates the presence of a particular sequence in the sample. Due to the large number of different probes present in a microarray, biological assays on microarrays can be conducted in a massively parallel fashion. Microarrays have therefore proven extremely useful in screening, profiling, and sequencing nucleic acid samples.

Assays conducted on microarrays typically employ fluorescently labeled targets. Fluorescent labels can provide high spatial resolution since the signal is generated by a species (i.e., the fluorescer) which is attached to the support either directly or through a probe-target interaction and which is therefore not free to migrate during the assay. In contrast to fluorophore-labeled targets, the use of enzyme labeled targets and chemiluminescent substrates results in a signaling species (i.e., the activated substrate) which is not attached to the support and which is therefore free to migrate during the assay. Migration of the chemiluminescent species during the assay can reduce the spatial resolution of the assay and can result in inaccurate assay data. As a result, chemiluminescent detection of enzyme labeled targets on microarrays has not been widely employed.

A need still exists, however, for improved methods of detecting chemiluminescent signals from solid supports, particularly from microarrays comprising higher feature density signal generating regions in applications involving multianalyte detection.

### SUMMARY

According to one embodiment of the invention, a method of detecting chemiluminescent emissions on a solid support is provided which includes contacting a surface layer of the solid support with a composition comprising a first chemiluminescent substrate capable of being cleaved by a first enzyme to produce a first chemiluminescent signal. First chemiluminescent signal on the surface layer of the solid support is then detected. The surface layer of the solid support is then contacted with a composition comprising a second chemiluminescent substrate capable of being cleaved by a second enzyme to produce a second chemiluminescent signal. Second chemiluminescent signal on the surface layer of the solid support is then detected. A plurality of probes are disposed in a plurality of discrete areas on the surface layer such that the density of discrete areas on the surface layer is at least 50 discrete areas/cm². At least some of the probes are bound to a first enzyme conjugate comprising the first enzyme and at least some of the probes are bound to a second enzyme conjugate comprising the second enzyme. Detection can be performed using any known detection device. Exemplary detection devices include a charge coupled device (i.e., a CCD) and a scanning system comprising a confocal microscope. The first and/or the second chemiluminescent substrates can also be contacted with the surface layer of the solid support in the presence of a chemiluminescent quantum yield enhancing material.

### DETAILED DESCRIPTION OF EMBODIMENTS

According to one embodiment of the invention, a process comprising the sequential generation of chemiluminescent signals provided. According to this embodiment of the invention, the support surface is contacted sequentially with different chemiluminescent (e.g., 1,2-dioxetane) substrates.

Since the signals are generated sequentially, the signal from each substrate can be detected without the use of optical filters. In fact, since detection is sequential rather than simultaneous, substrates having the, same emission maxima (e.g., substrates emitting radiation of the same wavelength or color) can be used. Also, buffers optimized for each enzyme can be used to maximize the emissions from each substrate.

Substrates having different emission maxima (e.g., substrates emitting radiation of different wavelength or color), however, can also be used. Further, when substrates having different emission maxima are used, filters can also be employed to further discriminate the emission wavelength of each substrate. The use of filters, however, is optional even when using substrates having different emission maxima.

Substrates having different light emission kinetics can be used for sequential detection. For example, the first substrate can give flash kinetics where the signal is collected within a period of seconds, during which time the signal diminishes from maximal signal to background. The second substrate can then be activated to give either flash or glow kinetics with light emission occuring over seconds, minutes or hours.

In another mode of sequential detection, oxyanion pKa control of dioxetane emission can be used. According to this embodiment, the first substrate used can emit light at an initial pH (e.g., at a pH of 7.0 using CDP-Star® substrate, TFE-CDP-Star® substrate or Galacton-Starg substrate, all of which are registered trademarks of Applied Biosystems, Foster City, Calif.) whereas the emissions from the second substrate (e.g., a nascent light emitting moiety) can build up from enzyme hydrolysis. According to this embodiment, the second enzyme substrate can be activated to emit light by increasing the pH (e.g., to a pH of 9 to 11).

A further advantage of sequential detection is that different buffers can be chosen for each substrate to optimize the chemiluminescent signal. Optimal buffers for both alkaline phosphatase and β-galactosidase are well known. For example, a standard buffer for alkaline phosphatase is a 0.1 M aminomethylpropanol solution comprising 1 mM MgCl₂ and having a pH of about 9.5. A standard buffer for β-galactosidase is a 0.1 M sodium phosphate solution comprising 1 mM MgCl₂ and having a pH of about 7.0.

A further advantage of sequential detection is that different enhancers, additives and/or counterions as disclosed in copending U.S. patent application Ser. No. 10/462,742 (Attorney Docket No. 9550-013-27), filed on Jun. 17, 2003, can be chosen to optimize the chemiluminescent signal for each substrate. Exemplary enhancers include, but are not limited to, poly(vinylbenzylammonium salts), poly(vinylbenzylphosphonium salts) and poly(vinylbenzylsulfonium salts). Exemplary counterions include, but are not limited to, halide (e.g., chloride or bromide), sulfate, alkylsulfonate, triflate, arylsulfonate, perchlorate, alkanoate, and arylcarboxylate.

According to one embodiment of the present invention, a method of detecting chemiluminescent emissions on a solid support is provided. The method comprises contacting a surface layer of the solid support with a composition comprising a first chemiluminescent substrate capable of being cleaved by a first enzyme to produce a first chemiluminescent signal. First chemiluminescent signal on the surface layer of the solid support is then detected. After detection of the first chemiluminescent signal, the surface layer is contacted with a composition comprising a second chemiluminescent substrate capable of being cleaved by a second enzyme to produce a second chemiluminescent signal. Second chemiluminescent signal on the surface layer of the solid support is then detected. A plurality of probes are disposed in a plurality of discrete areas on the surface layer. At least some of the probes are bound to a first enzyme conjugate comprising the first enzyme and at least some of the probes are bound to a second enzyme conjugate comprising the second enzyme. The first and second enzymes according to an embodiment of the invention are different. The surface layer can be washed after detection of the first chemiluminescent signal to remove chemiluminescent substrate activated by the first enzyme conjugate.

The composition comprising the first chemiluminescent substrate and the composition comprising the second chemiluminescent substrate can be contacted with the surface layer in the presence of a chemiluminescent enhancing material and/or a chemiluminescent enhancing additive. The use of chemiluminescent enhancing materials and chemiluminescent enhancing additives in solid phase chemiluminescent assays is disclosed in copending U.S. patent application Ser. No. 10/462,742 (Attorney Docket No. 9550-013-27), filed on Jun. 17, 2003, which application is incorporated by reference herein in its entirety. Any of the materials and techniques disclosed in this application can be used. For example, the chemiluminescent quantum yield enhancing material and/or enhancement additive can be incorporated into the solid support prior to contacting the solid support with the substrate. Alternatively, the chemiluminescent quantum yield enhancing material and/or enhancement additive can be included in the substrate composition. The chemiluminescent quantum yield enhancing material and/or enhancement additives can be selected based on the substrate being employed to optimize individual channels of signal detection.

Exemplary chemiluminescent quantum yield enhancing materials which can be used are disclosed in U.S. Pat. No. 5,145,772, which is hereby incorporated by reference in its entirety. Exemplary chemiluminescent enhancement additives which can be used are disclosed in U.S. Pat. No. 5,547,836, which is also hereby incorporated by reference in its entirety.

The first and second enzyme conjugates can each be bound indirectly to the probes on the support surface. For example, the first and second enzyme conjugates can be bound to first and second target molecules, respectively, wherein first and second target molecules are each bound to probes. According to this embodiment of the invention, the first and second enzyme conjugates can be antibody-enzyme conjugates wherein the first and second target molecules comprises an antigen moiety capable of being bound by the antibody. Alternatively, the first and second enzyme conjugates can each be bound directly to probes (i.e., the target molecules in the sample can be directly labeled with an enzyme).

According to another embodiment of the invention, the method as set forth above can further comprise contacting the support surface with a sample comprising first target molecules labeled with a first label and second target molecules labeled with a second label prior to contacting the support surface with the substrate composition. The first target molecules can be labeled with the first enzyme to form the first enzyme conjugate and the second target molecules can be labeled with the second enzyme to form the second enzyme conjugate. Alternatively, the first target molecules can be labeled with a moiety capable of binding to the first enzyme conjugate and the second target molecules can be labeled with a moiety capable of binding to the second enzyme conjugate. For example, the first and second enzyme conjugates can comprise enzyme-antibody conjugates and the first and second target molecules can be labeled with an antigen for the antibody.

The probes on the surface layer of the solid support can be oligonucleotide or nucleic acid probes. The first target molecules can be a first pool of target nucleic acids and the second target molecules be a second pool of target nucleic acids. The first and second pools of target nucleic acids can each comprise mRNA transcripts of one or more genes or nucleic acids derived from mRNA transcripts of one or more genes. For example, the first and second pools of target nucleic acids can each comprise cDNA or cRNA derived from mRNA transcripts. The concentration of the target nucleic acids in the first and second pools of target nucleic acids can be proportional to the expression level of the genes encoding the target nucleic acid.

Detecting the first chemiluminescent signal can comprise determining the location on the support surface of the first chemiluminescent signal and detecting the second chemiluminescent signal can comprise determining the location on the support surface of the second chemiluminescent signal.

Control probes can be located in one or more discrete areas on the support surface. For example, the control probes can be co-located in one or more of the same discrete areas as the analyte probes. The control probes can be used to normalize the data from the assay. For example, a known amount of a labeled control target can be added to the sample and the signal from the control target compared to the signal from the target molecules. The control target can be labeled with a fluorescent label or an enzyme label.

The support surface can also comprise a fluorescent label. The amount of a target molecule in a sample can be determined by comparing the intensity of the first and/or the second chemiluminescent signals to the intensity of the signal from the fluorescent label. The fluorescent label can also be used to determine the location of features (e.g., discrete areas) on the support surface. The fluorescent label can be imaged upon excitation (e.g., with an LED array) to localize the array elements and to provide data for the normalization of the quantitative chemiluminescence data from the array.

The surface layer of the solid support can be washed after contacting the support with the sample and before contacting the support with the first substrate composition. The sample is then incubated on the solid support to allow any labeled target molecules in the sample to bind to probes on the solid support. After the sample is incubated, the surface layer of the solid support can optionally be washed to remove any unbound material from the support surface. The target molecules in the sample can be labeled with an enzyme. Alternatively, the target molecules can be labeled with a moiety capable of specifically binding the first and second enzyme conjugates. The support surface can be contacted with a composition comprising the first and second enzyme conjugates after contact with the sample and before contact with the first substrate composition.

Chemiluminescent detection can be performed using any suitable detection technique. For example, chemiluminescence can be detected using a charge coupled device (i.e., a CCD).

As set forth above, the first and/or the second chemiluminescent substrates can be contacted with the surface layer of the solid support in the presence of a chemiluminescent quantum yield enhancing material. The chemiluminescent quantum yield enhancing material can be any of the materials disclosed in U.S. Pat. No. 5,145,772, which is hereby incorporated by reference in its entirety. Chemiluminescent enhancement additives may also be used to further improve the chemiluminescent signal. Exemplary chemiluminescent enhancement additives include any of the materials disclosed in U.S. Pat. No. 5,547,836, which is hereby incorporated by reference in its entirety. The chemiluminescent quantum yield enhancing material and/or enhancement additive can be incorporated into the solid support and/or added to a composition comprising the first or second substrates. See, for example, U.S. patent application Ser. No. 10/462,742, filed on Jun. 17, 2003, which application is incorporated herein by reference in its entirety.

As set forth above, some of the probes disposed on the support surface can be control probes. According to this embodiment of the invention, the sample can contain a known amount of an enzyme labeled control target and the substrate composition can contain a chemiluminescent substrate capable of being cleaved by the enzyme label on the control target (i.e., a control chemiluminescent substrate). Cleavage of the enzyme labile group on the control chemiluminescent substrate results in a chemiluminescent control signal. According to this embodiment of the invention, the amount of an analyte can be quantified by comparing the intensity of the chemiluminescent control signal to the intensity of a chemiluminescent signal derived from enzyme labeled analyte bound to the support surface. The location of the chemiluminescent control signal on the support surface can also be determined and used to locate features on the support surface.

A fluorescent control signal can also be used. According to this embodiment of the invention, the sample can contain a known amount of a fluorescent labeled control target. The amount of an analyte can be quantified by comparing the intensity of the fluorescent control signal to the intensity of a chemiluminescent signal from a labeled target molecule bound to the support surface. The location of the fluorescent control signal on the support surface can also be determined and used to locate features on the support surface. When a fluorescent control is used, two different chemiluminescent substrates can be used to sequentially assay two different analytes each labeled with a different enzyme.

The solid support surface can comprise a plurality of different probes each capable of binding with a different molecule. Groups of each of the probes can be disposed on the support surface in different discrete areas (e.g., in an array format). In this manner, the location of the signal on the surface of the solid support can be used to indicate the particular target molecule being detected. In the case of nucleic acid detection, the array can comprise a plurality of different oligonucleotide or nucleic acid probes capable of hybridizing to nucleic acids having substantially complementary nucleic acid sequences in the sample. According to this embodiment of the invention, detecting can comprise determining the location on the support surface of the chemiluminescent signals. The location of a chemiluminescent signal on the support surface can be determined using one or more enzyme labeled (e.g., chemiluminescent) or fluorescent control targets as set forth above.

If a control probe is used, the control probe can be located in one or more discrete areas on the support surface. For example, the control probe can be disposed in one or more discrete areas on the support surface either alone (i.e., in a discrete area comprising only control probes) or in combination with a probe for a target molecule (i.e., in a discrete area comprising both control and analyte probes).

The sample can comprise first target molecules comprising a first pool of target nucleic acids which is labeled directly with a first enzyme or with a moiety capable of binding a first enzyme conjugate and second target molecules comprising a second pool of target nucleic acids which is labeled directly with a second enzyme or with a moiety capable of binding a second enzyme conjugate. According to this embodiment of the invention, the target probes on the support surface can be oligonucleotide or nucleic acid probes. The first and second pools of target nucleic acids can each comprise mRNA transcripts of one or more genes or nucleic acids derived from the mRNA transcripts (e.g., cDNA or cRNA). The concentration of the target nucleic acids in the first and second pools of target nucleic acids can be proportional to the expression level of the genes encoding the target nucleic acid. In this manner, gene expression can be monitored and/or differences in gene expression between two pools of nucleic acids can be determined.

Although nucleic acid probes are described above, the analyte probes can also be polypeptides or any other molecule capable of binding or associating with a target biomolecule in a sample.

According to a further embodiment of the invention, the first chemiluminescent substrate and the second chemiluminescent substrate can emit chemiluminescent signals which are the same or different (i.e., wherein the differences in the emissions are detectable). For example, the emissions from the first and second chemiluminescent substrates can have the same or different emission maxima (i.e., can emit different colors).

Detection of the chemiluminescent signals can also be accomplished using filters (e.g., optical filters). For example, the second chemiluminescent signal can be detected by filtering the emissions from the support surface with a filter adapted to reduce the intensity of the first chemiluminescent signal relative to the intensity of the second chemiluminescent signal and detecting the first chemiluminescent signal. In this manner, residual chemiluminescence from contact of the support surface with the first chemiluminescent substrate can be reduced.

The composition comprising the first and/or the second chemiluminescent substrates can be a buffered solution. The buffer can be chosen to optimize detection (e.g., to maximize the emissions from each of the chemiluminescent substrates).

The methods described above can be applied to any solid support imaged with chemiluminescence. Exemplary solid supports that can be used include those disclosed in U.S. patent application Ser. No. 10/046,730, filed Jan. 17, 2002, pending, which application is incorporated herein by reference in its entirety. For example, the solid support may comprise an azlactone functional polymer layer. The solid support can be flexible, semi-rigid or rigid. Exemplary solid support materials include, but are not limited to, silicon, plastic, glass, membrane coated glass, nylon, nitrocellulose, polyethylsulfone, and pigment-impregnated variations thereof. The substrate may be porous or non-porous. Exemplary substrates include porous nylon and glass.

The solid support surface may be two-dimensional (i.e., substantially planar). Alternatively, the support surface may be non-planar. For example, the support surface may comprise undulations resulting from relaxation of the solid support to increase feature density as set forth in International Publication No. WO 99/53319, and U.S. Patent Application Publication Nos. US 2001/0053497 A1 and US 2001/0053527 A1 which publications are hereby incorporated by reference in their entirety.

As set forth above, the probes on the support may be arranged in an array format wherein a plurality of different probes are disposed in discrete areas on the surface of a solid support. The array can be a microarray having a plurality of probes disposed in a discrete area on the surface of a solid support at a relatively high density. The density of the discrete areas in which probes are disposed on the surface layer, for example, can be at least 50 discrete areas per cm², at least 100 discrete areas per Cm², at least 400 discrete areas per cm², at least 1,000 discrete areas per cm², at least 25,000 discrete areas per cm², or at least 50,000 discrete areas per cm².

For purposes of determining surface area, the projected (i.e., 2-dimensional) surface area and not the topographical (i.e., 3-dimensional) surface area of the solid support surface is used. The projected and topographical surface areas can differ significantly for solid support surfaces that are not macroscopically planar. For example, an undulated surface will have a topographical surface area that is greater than its projected (i.e., 2-dimensional) surface area. On the other hand, a macroscopically planar surface will have the same projected and topographical surface areas.

The density of a microarray can also be defined by the center to center distance between adjacent spots on the array which is commonly referred to as the “pitch” or the “probe pitch” of the array. The microarrays according to further embodiments of the invention can, for example, have probe pitches of 500 μm or less, of 300 μm or less, of 250 μm or less, or of 80 μm or less. The above ranges are exemplary and other ranges of probe pitch can also be used.

A control probe and/or a control label may be positioned in one or more of the same discrete areas on the support surface along with a probe for a target analyte. The signal from the control can be used to locate features on the array and/or to normalize the signal from the target analyte. Any of the types of controls disclosed in U.S. patent application Ser. No. 10/050,188, filed Jan. 14, 2002, pending, which is incorporated by reference herein in its entirety, may be used as a control. For example, a control label can be attached to a discrete area on the support surface via attachment of the control label directly to a probe for a target molecule or via attachment to a different molecule attached to the discrete area on the support surface along with the target probe. Alternatively, a control label can be attached to a control target capable of binding (e.g., hybridizing) to a control probe attached to one or more discrete areas on the support surface. The control target can be included in known quantity in the sample.

Any combination or one or more of the above types of controls can be used. For example, a control label and a control probe may both be attached to the support surface and the sample may include a control target (i.e., a target comprising a control label) capable of binding to the control probe. Additionally, the control label may be any type of label including an enzyme label (e.g., for a chemiluminescent substrate) or a fluorescent label.

Any chemiluminescent, enzyme-activatable compound can be used as a chemiluminescent substrate. For example, the chemiluminescent substrate can be a luminol, an acridan ester or thioester, an acridan enol phosphate or other enol phosphates, or a 1,2-dioxetane compound. The 1,2-dioxetane compound can be induced to decompose to yield a moiety in an excited state having a heteropolar character that makes it susceptible to environmental effects, particularly to dampening or diminution of luminescence in a polar protic environment. The chemiluminescent compound can be used to determine the presence, concentration or structure of a substance in a polar protic environment, particularly a substance in an aqueous sample.

Among the most effective compounds for this purpose are the stabilized, enzyme-cleavable 1,2-dioxetanes. A number of classes of these chemiluminescent enzyme-triggerable 1,2-dioxetanes, containing a variety of stabilizing functions are known. For example, spiro-bound polycycloalkyl groups either unsubstituted, substituted, or containing sp2 centers are taught in U.S. Pat. Nos. 5,112,960, 5,225,584, and 6,461,876, which are hereby incorporated by reference in their entirety. In addition, branched dialkyl-stabilized, enzyme-triggerable dioxetanes are taught in U.S. Pat. No. 6,284,899, which is also incorporated by reference in its entirety. Substituted furan and pyran-stabilized enzyme-triggerable dioxetanes are taught in U.S. Pat. No. 5,731,445, and European Patent Application Nos. EP 0943618 and EP 1038876, which are also incorporated by reference herein in their entirety. Any of the chemiluminescent substrates disclosed in the aforementioned patents and publications can be used.

A dioxetane having a stabilizing moiety can be used as a chemiluminescent substrate. The stabilizing moiety can be chosen based on the requirements of the application. Further, the dioxetanes may also be further substituted with one or more electron withdrawing (e.g. chlorine or fluorine), electron donating (e.g. alkyl or methoxy) groups, or deuterium atoms at any position. This allows tailoring of the quantum yield, emission half-life or pKa [Star dioxetanes] of the enzyme product. The dioxetane can be protected with an enzyme-labile group to form an enzyme cleavable substrate.

As set forth above, stabilized 1,2-dioxetanes (e.g., 1,2-dioxetanes stabilized with an adamantyl group) can be used as the chemiluminescent substrate. This class of dioxetanes can be represented by the following general formula: 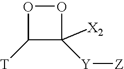
wherein T in the above formula represents an unsubstituted or substituted cycloalkyl, aryl, polyaryl or heteroatom group (e.g., an unsubstituted cycloalkyl group having from 6 to 12 ring carbon atoms, inclusive); a substituted cycloalkyl group having from 6 to 12 ring carbon atoms, inclusive, and having one or more substituents which can be an alkyl group having from 1 to 7 carbon atoms, inclusive, or a heteroatom group which can be an alkoxy group having from 1 to 12 carbon atoms, inclusive, such as methoxy or ethoxy, a substituted or unsubstituted aryloxy group, such as phenoxy or carboxyphenoxy, or an alkoxyalkyloxy group, such as methoxyethoxy or polyethyleneoxy, or a cycloalkylidene group bonded to the 3-carbon atom of the dioxetane ring through a spiro linkage and having from 6 to 12 carbon atoms, inclusive, or a fused polycycloalkylidene group bonded to the 3-carbon of the dioxetane ring through a spiro linkage and having two or more fused rings, each having from 5 to 12 carbon atoms, inclusive, e.g., an adamant-2-ylidene group.

The symbol Y represents a chromophoric group capable of producing a luminescent substance, which can emit light from an excited energy state upon dioxetane decomposition initiated by enzyme activation.

The symbol X₂ represents hydrogen or an alkyl, aryl, aralkyl, alkaryl, heteroalkyl, heteroaryl, cycloalkyl or cycloheteroalkyl group, e.g., a straight or branched chain alkyl group having from 1 to 7 carbon atoms, inclusive; a straight or branched chain hydroxyalkyl group having from 1 to 7 carbon atoms, inclusive, or an —OR group in which R is a C₁-C₂₀ unbranched or branched, unsubstituted or substituted, saturated or unsaturated alkyl, cycloalkyl, cycloalkenyl, aryl, aralkyl or aralkenyl group, fused ring cycloalkyl, cycloalkenyl, aryl, aralkyl or aralkenyl group, or an N, O or S hetero atom-containing group, or an enzyme-cleavable group containing a bond cleavable by an enzyme to yield an electron-rich moiety bonded to the dioxetane ring. According to one embodiment of the invention, X₂ can be a methoxy group or a trifluoroethoxy group (—OCH₂CF₃).

The symbol Z in the above formula represents an enzyme-cleavable group containing a bond cleavable by an enzyme to yield an electron-rich moiety bonded to the dioxetane ring, e.g., a bond which, when cleaved, yields an oxygen anion, a sulfur anion, a nitrogen anion, or an amido anion such as a sulfonamido anion.

An exemplary chemiluminescent substrate is the CDP-Star® substrate (Applied Biosystems, Foster City, Calif.) which is represented by the following chemical formula:

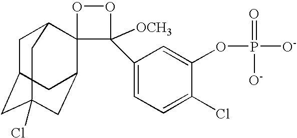

A further exemplary chemiluminescent substrate is the TFE-CDP-Star® substrate (Applied Bio systems, Foster City, Calif.) which is represented by the following chemical formula:

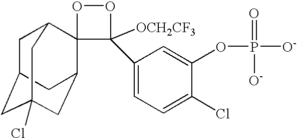

A further exemplary chemiluminescent substrate is Galacton-Star® substrate. Galacton-Star® is a registered trademark of Applied Biosystems, Foster City, Calif.

Deuterated dioxetanes can also be used as chemiluminescent substrates. Deuteration of the chemiluminescent dioxetane substrate can result in an increased chemiluminescent signal.

Chemiluminescent substrates other than dioxetanes can also be used. Exemplary chemiluminescent substrates include, but are not limited to, acridan ester or thioester substrates, acridan enol phosphate substrates, other enol phosphate substrates, and luminol substrates. When acridan ester or thioester substrates or luminol substrates are employed, the target molecules can be labeled with an oxidative enzyme such as a peroxidase (e.g., horseradish peroxidase), a catalase or a xanthine oxidase. Acridan enol phosphate and other enol phosphate substrates for alkaline phosphatase can also be used.

The first and second chemiluminescent substrates can both be 1,2-dioxetanes having different enzyme-cleavable groups. The first and second 1,2-dioxetane chemiluminescent substrates can emit the same or different chemiluminescent signals. In other words, first and second 1,2-dioxetanes chemiluminescent substrates can be identical except for the enzyme-cleavable group.

Alternatively, the first chemiluminescent substrate can be a 1,2-dioxetane chemiluminescent substrate and the second chemiluminescent substrate can be a non-dioxetane chemiluminescent substrate (e.g., an acridan or luminol substrate). According to this embodiment, each of the substrates can have a different enzyme-cleavable group (i.e., a group cleavable by a different enzyme).

Any type of probe that is capable of recognizing and binding to a target molecule in the sample can be used. Exemplary probes for nucleic acid targets include, but are not limited to, oligonucleotide probes and cDNA probes. For nucleic acid hybridization assays, the probe comprises a material that is capable of hybridizing with the target nucleic acid. Exemplary probes for protein or polypeptide targets include, but are not limited to, polypeptide probes, aptamer probes, and antibody probes.

The targets in the sample can be labeled with an enzyme capable of cleaving an enzyme labile group on a chemiluminescent substrate. Alternatively, the target can be labeled with a moiety capable of binding with an enzyme conjugate comprising an enzyme capable of cleaving an enzyme labile group on a chemiluminescent substrate. When the target is assayed indirectly, the target molecules can be labeled with a ligand and an enzyme conjugate capable of binding the ligand can be employed. Exemplary ligand/enzyme conjugate pairs which can be used include, but are not limited to, digoxigenin/antidigoxigenin:enzyme conjugates, biotin/streptavidin:enzyme conjugates, streptavidin/biotin:enzyme conjugates; and fluorescein/antifluorescein:enzyme conjugates.

Alternatively, the target can be unlabeled and detected by hybridization with a second labeled probe that binds to a portion of the target molecule different from that bound by the capture probe on the support surface. The second labeled probe can be labeled directly with an enzyme or with various ligands as set forth above and detected with an enzyme conjugate capable of binding the ligand.

Although the specific embodiments described above involve the sequential generation and detection of two chemiluminescent signals, additional chemiluminescent signals can also be used. Therefore, according to a further embodiment, three or more chemiluminescent signals can be sequentially detected.

The foregoing description is by way of example only and is not intended to be limiting. Although specific embodiments have been described herein for purposes of illustration, various modifications to these embodiments can be made without the exercise of inventive faculty. All such modifications are within the spirit and scope of the appended claims.

1. A method of detecting chemiluminescent emissions on a solid support, the method comprising:
contacting a surface layer of the solid support with a composition comprising a first chemiluminescent substrate capable of being activated by a first enzyme to produce a first chemiluminescent signal;
detecting first chemiluminescent signal on the surface layer of the solid support;
contacting the surface layer of the solid support with a composition comprising a second chemiluminescent substrate capable of being activated by a second enzyme to produce a second chemiluminescent signal; and
detecting second chemiluminescent signal on the surface layer of the solid support;
wherein a plurality of probes are disposed in a plurality of discrete areas on the surface layer at a density of at least 50 discrete areas per cm², wherein at least some of the probes are bound to a first enzyme conjugate comprising the first enzyme, and wherein at least some of the probes are bound to a second enzyme conjugate comprising the second enzyme.

2. The method of claim 1, wherein the composition comprising the first chemiluminescent substrate and the composition comprising the second chemiluminescent substrate are contacted with the surface layer in the presence of a composition comprising a chemiluminescent quantum yield enhancing material.

3. The method of claim 1, wherein the first and second enzyme conjugates are each bound indirectly to a probe.

4. The method of claim 3, wherein the first and second enzyme conjugates are bound to first and second target molecules, respectively, and wherein the first and second target molecules are each bound to a probe.

5. The method of claim 4, wherein the first and second enzyme conjugates comprise antibody-enzyme conjugates and wherein the first and second target molecules comprises an antigen moiety capable of being bound by the antibody.

6. The method of claim 1, wherein the first and second chemiluminescent substrates are 1,2-dioxetane substrates.

7. The method of claim 1, wherein the first and second enzyme conjugates are each bound directly to probes.

8. The method of claim 2, further comprising contacting the surface layer with the chemiluminescent quantum yield enhancing material before contacting the surface layer with the composition comprising the first chemiluminescent substrate.

9. The method of claim 1, wherein the density of discrete areas on the surface layer is at least 100 discrete areas per cm².

10. The method of claim 1, wherein the density of discrete areas on the surface layer is at least 1,000 discrete areas per cm².

11. The method of claim 1, wherein the density of discrete areas on the surface layer is at least 25,000 discrete areas per cm².

12. The method of claim 1, wherein the density of discrete areas on the surface layer is at least 50,000 discrete areas per cm².

13. The method of claim 1, further comprising:
contacting the support surface with a sample comprising, first target molecules labeled with a first label and second target molecules labeled with a second label prior to contacting the support surface with the substrate composition.

14. The method of claim 13, wherein the first target molecules are labeled with the first enzyme to form the first enzyme conjugate and the second target molecules are labeled with the second enzyme to form the second enzyme conjugate.

15. The method of claim 13, wherein the first target molecules are labeled with a moiety capable of binding to the first enzyme conjugate and the second target molecules are labeled with a moiety capable of binding to the second enzyme conjugate.

16. The method of claim 13, wherein the first target molecules comprise a first pool of target nucleic acids and wherein the second target molecules comprise a second pool of target nucleic acids.

17. The method of claim 16, wherein the first and second pools of target nucleic acids each comprise mRNA transcripts of one or more genes or nucleic acids derived from mRNA transcripts of one or more genes.

18. The method of claim 16, wherein the first and second pools of target nucleic acids each comprise cDNA or cRNA derived from mRNA transcripts.

19. The method of claim 17, wherein the concentration of the target nucleic acids in the first and second pools of target nucleic acids is proportional to the expression level of the genes encoding the target nucleic acid.

20. The method of claim 1, wherein the first and the second chemiluminescent substrates are each contacted with the surface layer in the presence of a chemiluminescent quantum yield enhancing material.

21. The method of claim 1, wherein detecting the first chemiluminescent signal comprises determining the location on the support surface of the first chemiluminescent signal and wherein detecting the second chemiluminescent signal comprises determining the location on the support surface of the second chemiluminescent signal.

22. The method of claim 21, wherein control probes are located in one or more discrete areas on the support surface.

23. The method of claim 22, wherein control probes are co-located in one or more of the same discrete areas as the analyte probes.

24. The method of claim 1, wherein the support surface further comprises fluorescent labels.

25. The method of claim 1, wherein the first chemiluminescent signal and the second chemiluminescent signal have different emission maxima.

26. The method of claim 25, wherein detecting the second chemiluminescent signal comprises:
filtering the emissions from the support surface with a filter adapted to reduce the intensity of the first chemiluminescent signal relative to the intensity of the second chemiluminescent signal; and
detecting the second chemiluminescent signal.

27. The method of claim 1, wherein the first chemiluminescent signal and the second chemiluminescent signal have approximately the same emission maxima.

28. The method of claim 1, wherein the composition comprising the first chemiluminescent substrate and the composition comprising the second chemiluminescent substrate are buffered compositions.

29. The method of claim 13, further comprising quantifying the amount of the first and the second target molecules in the sample.

30. The method of claim 29, wherein the support surface further comprises a fluorescent label and wherein quantifying comprises comparing the intensity of the first and/or the second chemiluminescent signals to the intensity of the signal from the fluorescent label.

31. The method of claim 1, further comprising washing the surface layer of the solid support after incubating and before contacting the surface layer with the first substrate composition.

32. The method of claim 1, further comprising washing the surface layer of the solid support after detecting the first chemiluminescent signal and before contacting the surface layer with the second chemiluminescent substrate composition.

33. The method of claim 1, wherein the first and second chemiluminescent substrates are both 1,2-dioxetanes.

34. The method of claim 1, wherein either of the first or second enzymes is β-galactosidase and the other enzyme is alkaline phosphatase.

35. The method of claim 34, wherein the composition comprising the chemiluminescent substrate capable of being activated by the alkaline phosphatase enzyme is a 0.1 M solution of aminomethylpropanol and 1 mM MgCl₂ at a pH of 9.5.

36. The method of claim 34, wherein the composition comprising the chemiluminescent substrate capable of being activated by the β-galactosidase enzyme is a 0.1 M solution of sodium phosphate and 1 mM MgCl₂ at a pH of 7.0.

37. The method of claim 15, further comprising contacting the support surface with a composition comprising the first and second enzyme conjugates.

38. The method of claim 37, wherein the first and second enzyme conjugates comprise enzyme-antibody conjugates and wherein the first and second target molecules are labeled with an antigen for the antibody.

39. The method of claim 1, wherein the first chemiluminescent substrate is a 1,2-dioxetane substrate and the second chemiluminescent substrate is selected from the group consisting of an acridan ester substrate, an acridan thioester substrate, an enol phosphate substrate, an acridan enol phosphate substrate, and a luminol substrate.

40. The method of claim 2, wherein the chemiluminescent quantum yield enhancing material is an onium polymer selected from the group consisting of poly(vinylbenzylammonium salts), poly(vinylbenzylphosphonium salts) and poly(vinylbenzylsulfonium salts).

41. The method of claim 2, wherein the chemiluminescent quantum yield enhancing material is an onium copolymer.

42. The method of claim 2, wherein the composition comprising the chemiluminescent quantum yield enhancing material further comprises an additive selected from the group consisting of BSA, cyclodextrins, negatively charged salts, alcohols, polyols, poly(2-ethyl-Z-oxazoline), zwitterionic surfactants, anionic surfactants, cationic surfactants, and neutral surfactants.

43. The method of claim 2, wherein the composition comprising the chemiluminescent quantum yield enhancing material further comprises counterion moieties selected from the group consisting of halide, sulfate, alkylsulfonate, triflate, arylsulfonate, perchlorate, alkanoate, arylcarboxylate and combinations thereof.

44. The method of claim 5, wherein the first or second enzyme conjugate is an antidigoxigenin:enzyme conjugate and wherein the corresponding target molecules are labeled with digoxigenin.

45. The method of claim 44, wherein the target molecules labeled with digoxigenin comprise cDNA.
